(19) United States
(12) Patent Application Publication
(10) Pub. No.: US 2016/0317207 A1
(43) Pub. Date: Nov. 3, 2016

(54) Cannulated Bone Tamp Device and Related Method Thereof

(71) Applicant: University of Virginia Patent Foundation, Charlottesville, VA (US)

(72) Inventors: Mark D. Miller, Crozet, VA (US); Christopher Little, Charlottesville, VA (US)

(21) Appl. No.: 15/141,385

(22) Filed: Apr. 28, 2016

Related U.S. Application Data
(60) Provisional application No. 62/153,772, filed on Apr. 28, 2015.
(60) Provisional application No. 62/327,642, filed on Apr. 26, 2016.

(51) Int. Cl. A61B 17/92; A61B 17/88

(52) U.S. Cl. CPC A61B 17/92; A61B 17/8897

(57) ABSTRACT

A system and method for conducting arthroscopic surgery. The system and related method may include: inserting a guide wire to a defect site; placing a lumen of a dowel over the guide wire to enable the dowel to travel toward the defect site; placing a guide pin over the guide wire to enable the guide pin to travel along the guide wire to be inserted into the lumen of the dowel; placing the cannula of a tamp over the guide pin to enable the tamp to travel along the guide pin toward the dowel; and impacting the tamp to apply a force to the tamp, wherein the tamp impacts the dowel to enable the dowel to advance with a predetermined fit-resistance into the defect site.

### RELATED APPLICATIONS

The present application claims benefit of priority under 35 U.S.C. §119 (e) from U.S. Provisional Application Ser. No. 62/153,772, filed Apr. 28, 2015, entitled “Cannulated Bone Tamp Device and Related Method Thereof” and U.S. Provisional Application Ser. No. 62/327,642, filed Apr. 26, 2016, entitled “Cannulated Bone Tamp Device and Related Method Thereof;” the disclosures of which are hereby incorporated by reference herein in their entirety.

### FIELD OF THE INVENTION

The present invention relates generally to the field of surgery directed to inserting bone 1.5 grafts to a target area. More specifically, the invention is in the subfield of inserting dense bone allograft cylinders into defects during revision anterior cruciate ligament (ACL) reconstruction.

### BACKGROUND

As anterior cruciate ligament (ACL) reconstruction is performed more frequently, the need for revision surgery is also increasing. Two-stage procedures for bony defects are commonly utilized, but carry the morbidity of two procedures. The present inventors recognize that the ability to perform that surgery in a single stage, regardless of previous tunnel position, helps to limit the morbidity and cost of this procedure. The present disclosure and related study demonstrates satisfactory clinical and radiographic outcomes after single stage revision ACL reconstruction.

### Overview

Revision anterior cruciate ligament (ACL) reconstruction involves removing a prior failed graft and replacing it with a new graft. This can be done in a one-step or two-step process, for example, based on several technical considerations. When a failed ACL graft and/or prior hardware is removed, there is often a residual defect in the bone. These defects can sometimes be quite large (as much as 18-mm in diameter, for example) through a process called osteolysis. An aspect of an embodiment of the present invention provides the ability to fill these defects a with bone graft prior to proceeding with revision ACL reconstruction. For example, an allograft “Cloward” bone dowels, that are used for cervical fusion, may be utilized. These grafts are acellular, can be easily stored for long periods, and readily available in different sizes. Other types of dowels (and respective materials) may be utilized as desired or required. An aspect of an embodiment of the present invention provides the ability to insert the dowels arthroscopically over a guide wire. The dowels may be inserted by use of an embodiment of the present invention cannulated tamp.

An aspect of an embodiment may include the steps, for example, as discussed below. Arthroscopically over drill a defect site from a prior ACL reconstruction. For example, over drill the defect site to the appropriate size. Arthroscopically place the guide pin (and/or guide wire) into said defect site. Arthroscopically have the lumen of a dowel (or other hardware or implant) placed over the guide pin to enable dowel (or other hardware or implant) to travel along the guide pin to the defect site. Alternatively, a guide wire may arthroscopically passed to the defect site to provide for the dowel (or other hardware or implant) to be arthroscopically inserted over a guide wire to the defect site. For example, a guide wire may initially be used to place the dowel (or other hardware or implant), and subsequently the guide pin is arthroscopically passed over the guide wire and then the guide pin can be inserted into the lumen (or to the lumen) of the dowel (or other hardware or implant) as the guide pin travels along the guide wire. The guide wire may be removed at any predetermined or given time.

Arthroscopically (or non-arthroscopically) place a cannulated tamp over the guide pin (or guide wire) to allow tamp to travel along the guide pin. Arthroscopically (or non-arthroscopically) using a tool or device (e.g, hammer) or the like, tap or advance the cannulated tamp behind the dowel to be able to force or advance the dowel (e.g., cylindrical bone graft) with proper fit-resistance into defect site.

An aspect of an embodiment of the present invention provides, but not limited thereto, a method for conducting arthroscopic surgery. The method may comprise: inserting a guide wire to a defect site; placing a lumen of a dowel over the guide wire to enable the dowel to travel toward the defect site; placing a guide pin over the guide wire to enable the guide pin to travel along the guide wire to be inserted into the lumen of the dowel; placing the cannula of a tamp over the guide pin to enable the tamp to travel along the guide pin toward the dowel, whereby the tamp having a proximal end and a distal end; and impacting the tamp to apply a force to the proximal end of the tamp wherein the tamp at its distal end impacts the dowel to enable the dowel to advance with a predetermined fit-resistance into the defect site.

An aspect of an embodiment of the present invention provides, but not limited thereto, a tamp device configured for conducting arthroscopic surgery. The tamp device may comprise: a proximal end, distal end, and a cannula longitudinally extending from proximal end to distal end, wherein: the tamp is configured wherein it's cannula is configured to slide over a guide pin and/or guide wire; and the tamp is configured to receive impact force at its proximal end so as transfer the force to a dowel to impact and advance the dowel to a defect site with predetermined fit resistance.

An aspect of an embodiment of the present invention provides, but not limited thereto, a kit for use in arthroscopic surgery for using a tamp. The kit may comprise: a tamp device comprising a proximal end, distal end, and a cannula longitudinally extending from proximal end to distal end, and instructions for use of the tamp. The instructions may include the following steps: inserting a guide wire to a defect site; placing a lumen of a dowel over the guide wire to enable the dowel to travel toward the defect site; placing a guide pin over the guide wire to enable the guide pin to travel along the guide wire to be inserted into the lumen of the dowel; placing the cannula of a tamp over the guide pin to enable the tamp to travel along the guide pin toward the dowel; and impacting the tamp to apply a force to the proximal end of the tamp wherein the tamp at its distal end impacts the dowel to enable the dowel to advance with a predetermined fit-resistance into the defect site.

An aspect of an embodiment of the present invention provides, but not limited thereto, a method for conducting arthroscopic surgery. The method may comprise: placing an implant at a defect site; placing a guide pin to be disposed on the implant; placing the cannula of a tamp over the guide pin to enable the tamp to travel toward the implant, the tamp having a proximal end and a distal end; and impacting the tamp to apply a force to the proximal end of the tamp wherein the tamp at its distal end impacts the implant to enable the implant to advance with a predetermined fit-resistance into the defect site.

An aspect of an embodiment of the present invention provides, but not limited thereto, a kit for use in arthroscopic surgery for using a tamp. The kit may comprise: a tamp device comprising a proximal end, distal end, and a cannula longitudinally extending from proximal end to distal end, and instructions for use of the tamp. The instructions may include the following steps: placing an implant at a defect site; placing a guide pin to be disposed on the implant; placing the cannula of a tamp over the guide pin to enable the tamp to travel toward the implant,; and impacting the tamp to apply a force to the proximal end of the tamp wherein the tamp at its distal end impacts the implant to enable the implant to advance with a predetermined fit-resistance into the defect site.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings.

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 provides a perspective schematic view of the tamp having a handle (or the like).

FIG. 2 provides an enlarged partial view of the tamp of FIG. 1.

FIG. 3 provides an enlarged partial view of the tamp of FIG. 1.

FIG. 4 provides a perspective schematic view of the guide pin.

FIG. 5 provides a perspective schematic view of a dowel with a lumen slidably disposed on the guide pin.

FIG. 6 provides a perspective schematic view of a tamp with a cannula slidably disposed on the guide pin, for example as the one shown in FIGS. 4 and 5.

FIG. 7 provides a perspective schematic view of a tamp having been advanced, driven or pounded into the dowel of FIG. 6.

FIG. 8 provides a perspective schematic view of the guide pin.

FIG. 9 provides a perspective schematic view of a dowel with a lumen slidably disposed on the guide pin.

FIG. 10 provides a perspective schematic view of a tamp with a cannula slidably disposed on the guide pin, for example as the one shown in FIGS. 8 and 9.

FIG. 11 provides a perspective schematic view of a tamp having been advanced, driven or pounded into the dowel of FIG. 10.

FIG. 12 provides a perspective schematic view of a tamp with a cannula disposed on the guide pin.

FIG. 13 provides a perspective schematic view of a guide pin and a cannulated tamp.

FIG. 14 provides a perspective schematic view of the guide pin disposed inside the cannulated tamp via its cannula.

FIG. 15 provides a top perspective schematic view of the tamp (as shown in FIGS. 13 and 14, for example) whereby its distal end is in contact with a dowel.

FIGS. 16(A)-(B) provide arthroscopic images of single-stage ACL revision reconstruction.

FIGS. 17(A)-(B) provide CT images of a 18-year-old woman following ACL revision reconstruction utilizing bone-patellar tendon-bone graft.

FIGS. 18(A)-(B) provide CT images of a 21-year-old man 18 months following single-stage ACL revision reconstruction with patellar tendon autograft and 12 mm diameter allograft bone dowel for failed previous hamstring autograft ACL reconstruction as reflected in the angled oblique coronal CT reformatted image and angled axial CT image, respectively.

### DETAILED DESCRIPTION OF EXEMPLARY EMBODIMENTS OF THE INVENTION

The present inventors have developed and disclosed herein, but not limited thereto, a method and related device for inserting dense bone allograft cylinders (or other hardware or implants) into defects during revision anterior cruciate ligament (ACL) reconstruction. The present inventors have created, but not limited thereto, a cannulated tamp that can be used to tap these grafts into same-sized defects.

An aspect of an embodiment of the present invention provides, but not limited thereto, a pin that can be placed, and then the cannulated tamp can be placed over the pin so as to be able to tap in the cylindrical bone graft. For example, the grafts are tamped and fitted to achieve different degrees of fixation and interference fit.

An aspect of an embodiment of the present invention provides, among other things, a 3/32″ guide pin (or other size as desired or required), which may be used in ACL reconstruction, that is placed into a defect from a prior ACL reconstruction and is overdrilled to the appropriate size. A similar sized bone dowel is selected and a 3/32 drill hole is placed in the center of the dowel. An aspect of an embodiment of the present invention dowels include drill hole or the like that extends through the entire length of the graft. The dowel can then be gently tapped into place.

It should be appreciated that the tamp, pin, cannulations, drill holes, and dowels may be a variety of widths and lengths as desired or required for anatomical and operational purposes. Complimentary size drill bits (and other tools and devices) of varying lengths and widths may be utilized accordingly.

It should be appreciated that an embodiment of the present invention fixation device may be comprised of a variety of materials as desired or required.

It should be appreciated that various sizes, dimensions, contours, rigidity, shapes, flexibility and materials of any of the components or portions of components in the various embodiments of the device discussed throughout may be varied and utilized as desired or required. Similarly, locations and alignments of the various components may vary as desired or required.

It should be appreciated that any of the components or modules referred to with regards to any of the present invention embodiments of the device discussed herein, may be integrally or separately formed with one another. Further, redundant functions or structures of the components or modules may be implemented.

It should be appreciated that the device and related components of the device discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

Further, it should be appreciated that an embodiment may be implemented and directed at other target areas or intended areas in addition to as disclosed and discussed.

It should be appreciated that the patient or subject receiving surgery is intended to be a human. However, it should be appreciated that various embodiments may be directed to an animal and may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc.

An aspect of various embodiments of the present invention may be utilized for a number of products and services, such as but not limited thereto, the following: use for ACL revision surgery.

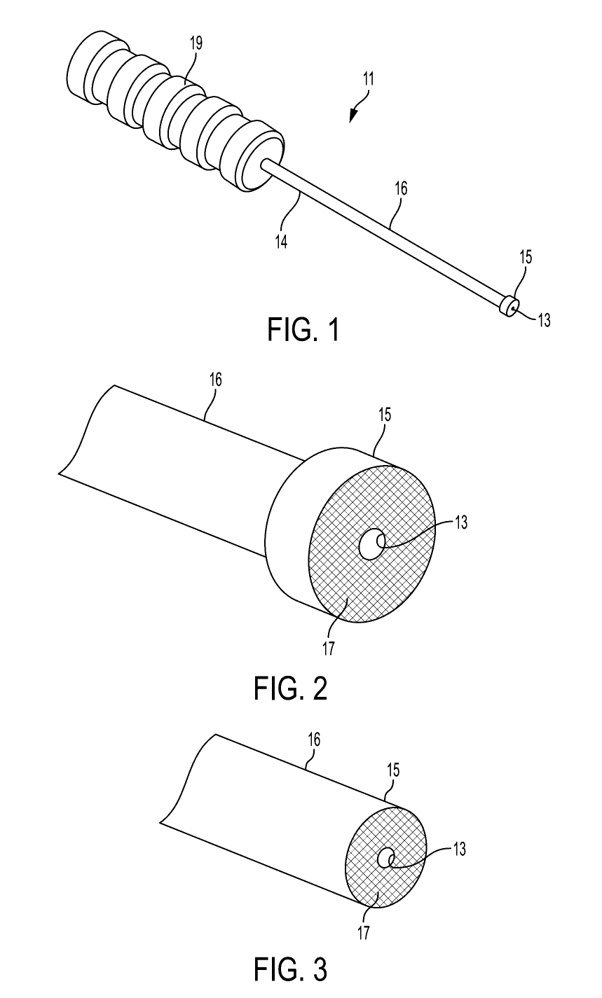

FIG. 1 provides a perspective schematic view of the tamp 11 having a handle 19 (or the like) at the proximal end 14, a shaft 16, a cannula 13 disposed therein, and a distal end 15. In this particular embodiment the distal end 15 is shown with a flange configuration. The cannula 13 may be configured with varying lengths so as to travel partially through the shaft 16, all the way through the shaft 16, through the shaft 16 and terminating within the handle 19, or all the way through the shaft and handle 19. The cannula 13 may be configured to receive a guide wire, guide pin or other medical devices, hardware or implants.

FIG. 2 provides an enlarged partial view of the tamp 11 of FIG. 1 illustrating the shaft 16, a cannula 13 disposed therein, and a distal end 15. In this particular embodiment, the distal end 15 is shown with a flange configuration. The flange of the distal end 15 has a face 17 with a predetermined surface, texture, material, or configuration. In an embodiment, it is provided generally as a flat surface. The face 17, may for example, but not limited thereto, have a abrasive surface or other surface for traction (to avoid slippage

FIG. 3 provides an enlarged partial view of the tamp 11 of FIG. 1 illustrating the shaft 16, a cannula 13 disposed therein, and a distal end 15. In this particular embodiment the distal end 15 is shown without a flange configuration.

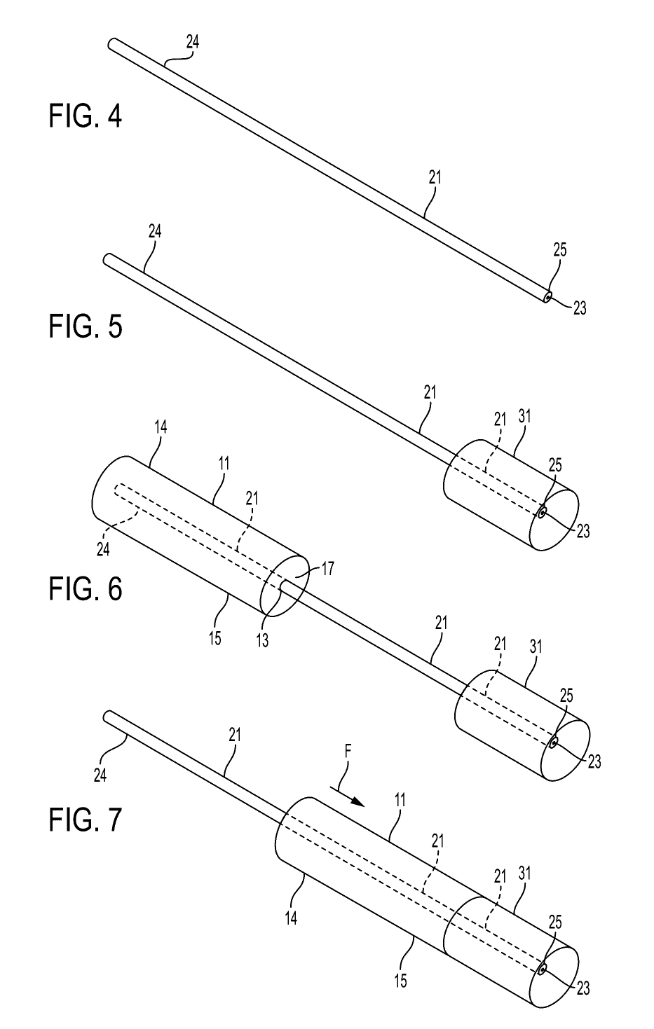

FIG. 4 provides a perspective schematic view of the guide pin 21 having a proximal end 24 and a distal end 25. During use, the surgeon or user is able place the distal end 25 of guide pin 21 into the defect site (not shown) or target area of the subject, or other required or intended area or location. Alternatively, during use, the surgeon or user is able to place the distal end 25 of guide pin 21 over a guide wire or other medical device to be disposed in the lumen 23 of the guide pin 21, which may already be in the defect site (not shown) or target area of the subject, or other required or intended area or location. Alternatively, during use, the surgeon or user is able to place the distal end 25 of guide pin 21 over a guide wire or other medical device to be disposed in the lumen 23 of the guide pin 21, and which will eventually be placed at the defect site (not shown) of the subject, or intended area or location at a predetermined or given time.

FIG. 5 provides a perspective schematic view of a dowel 31 with a lumen 33 slidably disposed on the guide pin 21, for example as the one shown in FIG. 4. The Lumen 33 of the dowel 31 transverses the entire length of the dowel 31. During use, the surgeon or user can place the lumen 33 of a dowel 31 over the guide pin 21 to enable dowel 31 to travel along the guide pin 21 to or toward the defect site (not shown) of the subject, or other required or intended area or location. Alternatively, during use the surgeon or user can place the lumen 33 of a dowel 31 over a guide wire (not shown). Thereafter, the guide pin 21 can be slid over the guide wire (not shown). Thereafter, the guide pin 21 can travel to the dowel 31; or the guide pin 21 can travel to inside the lumen of the dowel 31 so that the guide pin 21 and dowel 31 can travel to or toward the defect site (not shown) of the subject, or other required or intended area or location.

In an alternate embodiment, the lumen 33 may only partially pass through the length of the dowel 31, such as providing access at one end of the dowel 31 and which terminates within the dowel instead of passing completely there through the dowel 31.

FIG. 6 provides a perspective schematic view of a tamp 11 with a cannula 13 slidably disposed on the guide pin 21, for example as the one shown in FIGS. 4 and 5. The tamp 11 has a proximal end 14 and a distal end 15. The distal end 15 having a face 17 with a predetermined surface, texture, material, geometry, contours, or configuration. During use the surgeon or user can place the cannula 13 of the tamp 11 over the guide pin 21 to allow tamp 11 to travel along the guide pin 21 to a location(s) as on the guide pin 21 as desired or required. Alternatively, during use, the surgeon or user can place the cannula 13 of the tamp 11 over the guide wire (not shown) or other medical device to allow tamp 11 to travel along the guide wire or other medical device, to a location(s) along the guide wire as desired or required.

FIG. 7 provides a perspective schematic view of a tamp 11 having been advanced, driven or pounded into the dowel 31 with a desired, required, or predetermined force applied to the tamp 11, as schematically illustrated by arrow, F. Resultantly, the force, F, exerted on the tamp 11 is transferred to the dowel 31. Accordingly, the surgeon or user is able to advance the dowel 31 with proper fit-resistance into defect site of the subject (or intended target area). For example, using a impact device or hammer (not shown), surgical tool, or the like, the surgeon or user taps or pounds the handle 19 of the tamp 11 into and behind the dowel 31 so that the dowel 31 is driven to the intended location and with the intended fit-resistance with the defect site (or other intended site, area or location) of the subject.

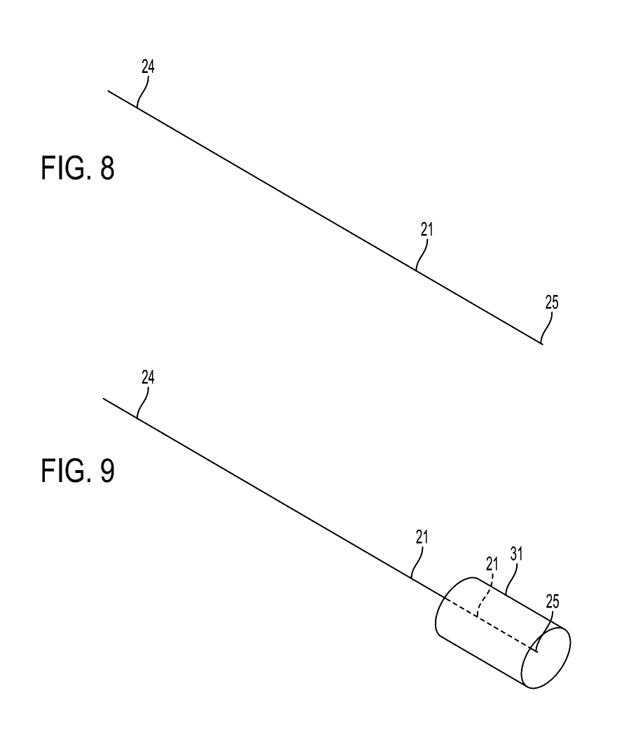

FIG. 8 provides a perspective schematic view of the guide pin 21 having a proximal end 24 and a distal end 25. During use, the surgeon or user is able place the distal end 25 of guide pin 21 into the defect site (not shown) or target area of the subject, or other required or intended area or location. Alternatively, during use, the surgeon or user is able to place the distal end 25 of guide pin 21 over a guide wire or other medical device to be disposed in the lumen (not shown) of the guide pin 21, which may already be in the defect site (not shown) or target area of the subject, or other required or intended area or location. Alternatively, during use, the surgeon or user is able to place the distal end 25 of guide pin 21 over a guide wire or other medical device to be disposed in the lumen (not shown) of the guide pin 21, and which will eventually be placed at the defect site (not shown) of the subject, or intended area or location at a predetermined or given time.

FIG. 9 provides a perspective schematic view of a dowel 31 with a lumen 33 slidably disposed on the guide pin 21, for example as the one shown in FIG. 8. The Lumen 33 of the dowel 31 transverses the entire length of the dowel 31. During use the surgeon or user can place the lumen 33 of a dowel 31 over the guide pin 21 to enable dowel 31 to travel along the guide pin 21 to or toward the defect site (not shown) of the subject, or other required or intended area or location. Alternatively, during use the surgeon or user can place the lumen 33 of a dowel 31 over a guide wire (not shown). Thereafter, the guide pin 21 can be slid over the guide wire (not shown). Thereafter, the guide pin 21 can travel to the dowel 31; or the guide pin 21 can travel to inside the lumen of the dowel 31 so that the guide pin 21 and dowel 31 can travel to or toward the defect site (not shown) of the subject, or other required or intended area or location.

In an alternate embodiment, the lumen 33 may only partially pass through the dowel 31, such as providing access at one end of the dowel 31 and which terminates within the dowel instead of passing completely there through the dowel 31.

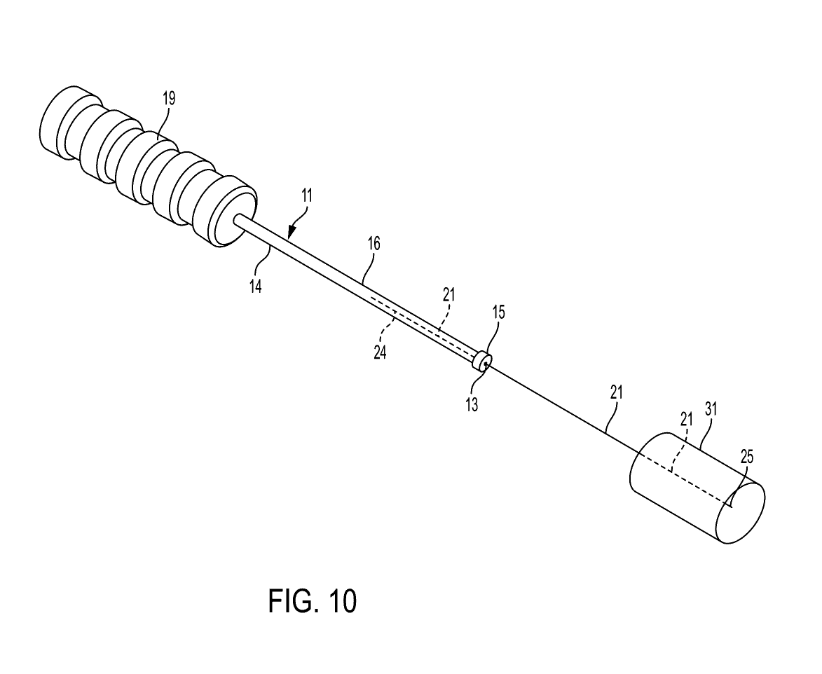

FIG. 10 provides a perspective schematic view of a tamp 11 including its shaft 16 with a lumen 13 slidably disposed on the guide pin 21, for example as the one shown in FIGS. 4 and 5. The tamp 11 has, for example, a proximal end 14, a distal end 15, a shaft 16, and a handle 19. The distal end 15 having a face 17 with a predetermined surface, texture, material, geometry, contours, or configuration. During use the surgeon or user can place the cannula 13 of the tamp 11 over the guide pin 21 to allow tamp 11 to travel along the guide pin 21 to a location(s) as on the guide pin 21 as desired or required. Alternatively, during use the surgeon or user can place the cannula 13 of the tamp 11 over the guide wire (not shown) or other medical device to allow tamp 11 to travel along the guide wire or other medical device, to a location(s) along the guide wire as desired or required.

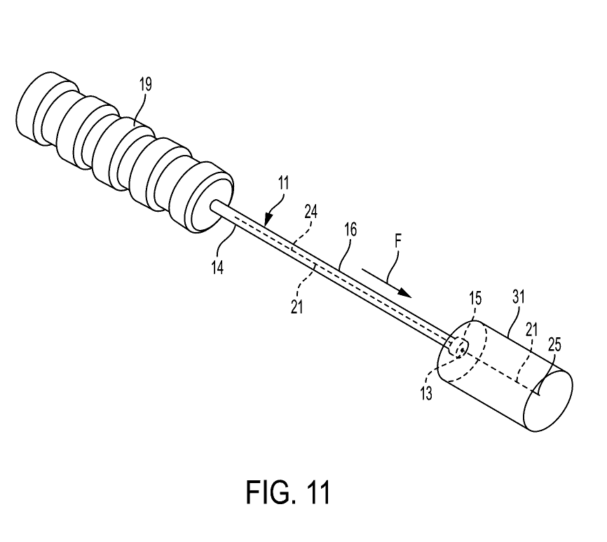

FIG. 11 provides a perspective schematic view of a tamp 11 including its shaft 16 having been advanced, driven or pounded into the dowel 31 with a desired, required, or predetermined force applied to the handle 19 of the tamp 11, as schematically illustrated by arrow, F. Resultantly, the force, F, exerted on the tamp 11 is transferred to the dowel 31. Accordingly, the surgeon or user is able to advance the dowel 31 with proper fit-resistance into defect site of the subject (or intended target area). For example, using a hammer (not shown), surgical tool or the like, the surgeon or user taps or pounds the proximal end 14 (for example at the handle 19) of the tamp 11 into and behind the dowel 31 so that the dowel 31 is driven to the intended location and with the intended fit-resistance with the detect site (or other intended site, area or location) of the subject.

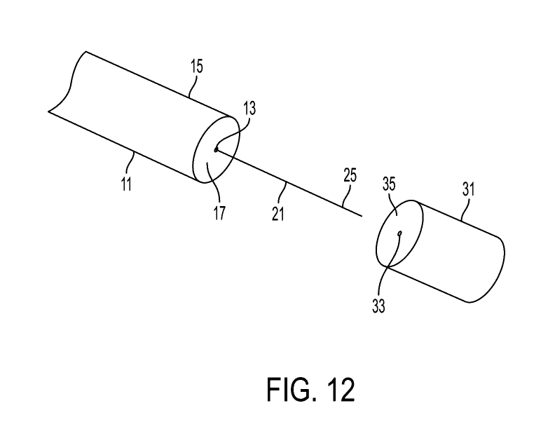

FIG. 12 provides a perspective schematic view of a tamp 11 (partial view) with a cannula 13 slidably disposed on the guide pin 21, wherein the guide pin 21 having a proximal end 24 (not shown, as it's already disposed inside the tamp 11) and a distal end 25. Regarding the present illustrative example, the surgeon or user has not yet placed the guide pin 21 into the dowel 31, which may include a lumen 33 and a proximal end 35 of the lumen 31.

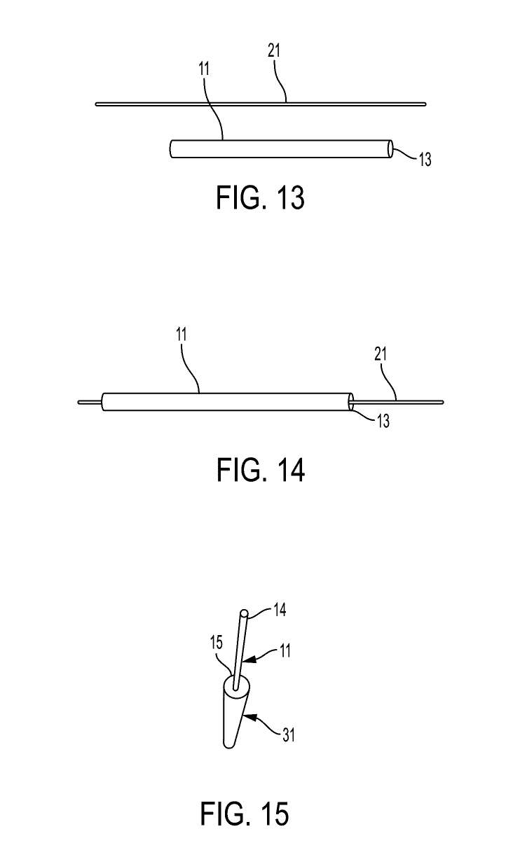

FIG. 13 provides a perspective schematic view of a guide pin 21 and a cannulated tamp 11.

FIG. 14 provides a perspective schematic view of the guide pin 21 slidably disposed inside the cannulated tamp 11 via its cannula 13.

FIG. 15 provides a top perspective schematic view of the tamp 11 (as shown in FIGS. 13 and 14, for example) having a proximal end 14 and whereby its distal end 15 is in contact with a dowel 31.

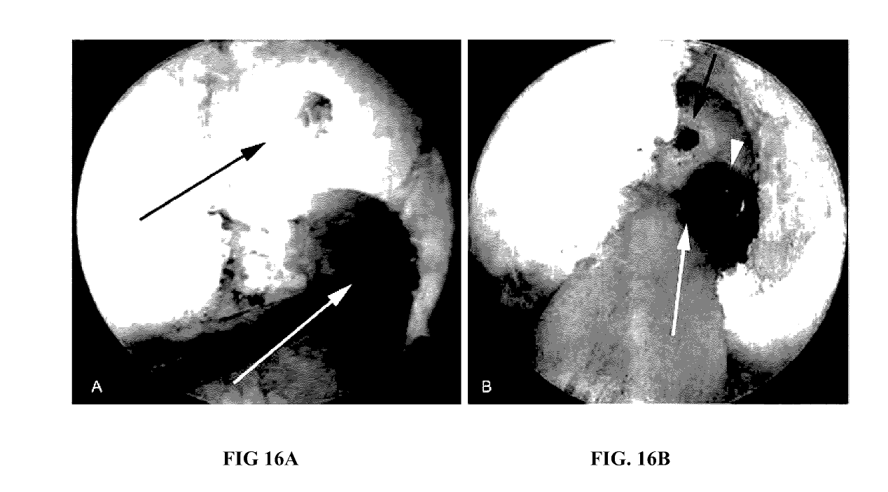

FIG. 16 provides arthroscopic images of single-stage ACL revision reconstruction. Referring to FIG. 16(A), the image demonstrates new tunnel (white arrow) in relation to the new allograft dowel graft (black arrow) occupying the old malpositioned tunnel. Referring to FIG. 16(B), the image demonstrates metal interference screw (white arrowhead) with purchase on surrounding native bone, autograft bone from bone-patellar tendon bone graft (white arrow), and allograft bone dowel (black arrow) within old tunnel.

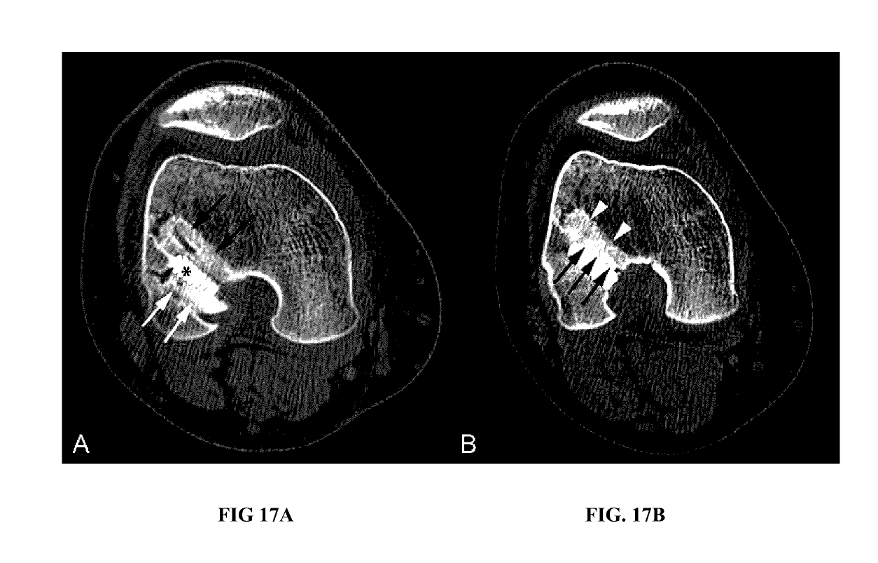

FIG. 17 provides CT images of a 18-year-old woman following ACL revision reconstruction utilizing bone-patellar tendon-bone graft. Referring to FIG. 17(A), the oblique axial CT image shows metal interference screw (asterisk) threads engaged with both the patellar bone graft (white arrows) and the allograft dowel (black arrows) filling the original tunnel from the first reconstruction. The dowel is integrated (black arrows) with the adjacent native bone and is intact except for mild reduction from the pathway of the revision tunnel and screw. Referring to FIG. 17(B), the oblique axial CT image shows excellent screw thread engagement (black arrows) with allograft dowel (arrowheads). The dowel is intact and shows excellent integration (arrowheads) with the surrounding bone.

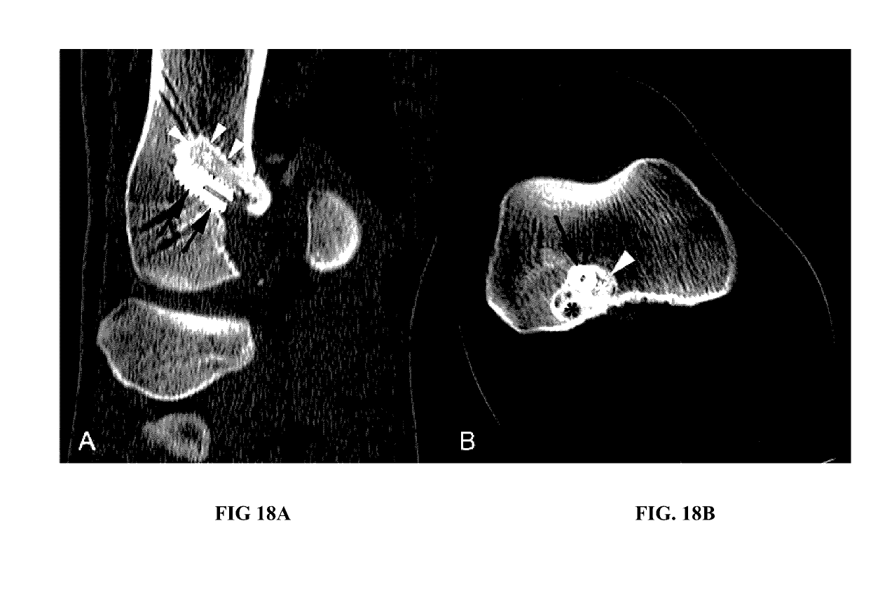

FIG. 18 provides CT images of a 21-year-old man 18 months following single-stage ACL revision reconstruction with patellar tendon autograft and 12 mm diameter allograft bone dowel (white asterisk) for failed previous hamstring autograft ACL reconstruction. Referring to The angled oblique coronal CT reformatted image (see FIG. 18(A)) and angled axial CT image (see FIG. 18(A)) demonstrate a metal interference screw (black arrows) with thread purchase on native femoral bone, allograft bone dowel, and bone from bone-patellar tendon-bone autograft (black asterisk). The dowel is well incorporated into native bone at all interfaces (white arrowheads). The dowel has been reduced in size, more evident in image (see FIG. 18(A)), from Obliquely oriented partial drilling prior to screw placement. Ghost tracks from the original reconstruction with hamstring tendon are incidentally evident (see FIG. 18(A)).

### EXAMPLES

Practice of an aspect of an embodiment (or embodiments) of the invention will be still more fully understood from the following examples and experimental results, which are presented herein for illustration only and should not be construed as limiting the invention in any way.

### Example and Experimental Results Set No. 1

Background: Two-stage techniques for revision ACL reconstruction are time consuming and technically demanding. Recently, single-stage techniques have been described in technical reports to avoid the potential morbidity of two-stage ACL revisions. The goal of this study is to is present clinical results of a series of patients who underwent single-stage revision ACL reconstruction utilizing an allograft bone dowel for an isolated femoral bony deficiency.

Methods: 16 patients who underwent single-stage revision ACL reconstruction utilizing an allograft bone dowel for an isolated femoral bony deficiency between 2007 and 2012 were identified. 12 patients completed study visits, resulting in 75% follow-up at an average of 2.6 years. Study visits included computed tomography (CT) scans as well as completion of validated outcomes measures. Physical examination and a KT-1000 exam were also performed.

Results: The average KT-1000 side-to-side difference was 1.0 mm±2.9 mm. The average IKDC was 70.2±17.8, the average Tegner activity score was 4.8±2.8 and the average VAS pain score was 2.8±2.4, The overall average KOOS was 70.5±22.4. The average KOOS subscale scores were: KOOS pain: 73.4±22.3, KOOS Symptoms: 67.3±20.0, KOOS ADLs: 82.8±22.9, KOOS Sports 50.8±31.3 and KOOS QOL: 41.2±26.1. Analysis of CT scans found that 100% (12/12) dowels had “excellent” (greater than 75%) incorporation. There were no instances of osteolysis or cyst formation noted. The mean relative density of the 12 dowels was 607.3 HU±105.6 HU.

Conclusions: A single-stage approach for revision ACL reconstruction utilizing an allograft dowel for isolated femoral bony deficiency yields Objective and subjective outcomes comparable to those reported in the literature for two-stage and other single-stage techniques. The allograft bone dowels described in the study demonstrate the ability to integrate with the host bone, adequately fill bony defects from previous tunnels and withstand the stresses required for fixation and stability of ACL grafts.

### Introduction

Anterior cruciate ligament (ACL) rupture is a common athletic injury, with a reported incidence of 37 to 61 per 100,000 person years. The number of primary ACL reconstructions has increased considerably over the past twenty years, with over a 30% increase in incidence from 32.9 to 43.5 reconstructions per 100,000 person-years in the United States alone. While the majority of patients fare quite well following primary ACL reconstruction, between 1.7% and 9.4% of patients will eventually require revision ACL reconstruction.

Revision ACL surgery must address any modifiable factors contributing to the primary failure. 24% of failures can be entirely attributed to technical error, with tunnel mal positi on being the most common. More than 70% of technical failures and 50% of all ACL failures can be attributed to malpositioned tunnels. Other factors contributing to ACL reconstruction failure include graft choice, patient activity, patient age, subsequent trauma, patient mechanics, and less commonly, presence of additional biologic pathology such as infection or inflammatory arthropathy.

Revision ACL reconstruction is substantially more challenging than a primary procedure, as addressing the source of technical error most often requires a change in tunnel position on the femoral side, tibial side, or both. Correct, anatomic tunnel placement is crucial in revision ACL reconstruction to prevent future failure and must be accomplished regardless of considerations of previous tunnel placement. Management of previously malpositioned or widened tunnels can be technically demanding and often requires innovative approaches for dealing with bony defects. Traditionally, large bone voids greater than 10 to 15 mm in diameter have been addressed using a two-stage procedure including initial bone grafting of the old tunnel(s) followed by delayed ligament reconstruction once the graft had incorporated, typically 6 weeks to 6 months later.

Two-stage techniques are time consuming, technically demanding, and subject the patient to the risk of two surgical procedures. Recently, single-stage techniques have been described in technical reports to avoid the potential morbidity of two-stage ACL revisions. The present inventor presents clinical results of a series of patients who underwent single-stage revision ACL reconstruction utilizing an allograft bone dowel for an isolated femoral bony deficiency.

### Methods

### Patients

After approval was obtained from the Institutional Review Board, a retrospective chart review of all patients who underwent revision ACL reconstruction at our institution between 2007 and 2012 was performed to identify a cohort of patients who underwent single stage revision ACL reconstruction utilizing an allograft bone dowel for an isolated femoral bony deficiency. Inclusion criteria were patients 1) revision ACL reconstruction, 2) isolated femoral bony deficiency requiring grafting, 3) use of an allograft bone dowel (Cloward dowel, LifeNet Health, Virginia Beach, Va.) and 4) age between 18 and 40 years. Exclusion criteria were 1) significant arthritic changes (Grade III or IV) noted on preoperative x-ray or during arthroscopy, 2) use of a bone dowel or graft other than a Cloward dowel, 3) presence of any tibial bony defect requiring the use of bone graft and 4) concomitant meniscus repair.

The medical records of all patients meeting inclusion and exclusion criteria were retrospectively reviewed. Demographic data, including age, gender, BMI, smoking status, date of initial and revision surgery and type of graft for initial and revision surgery were collected. Documentation from follow-up visits was also reviewed.

Patients were recruited for in-person study visits. Study visits included computed tomography (CT) scans of the operative knee to assess integration of the allograft dowel as well as completion of several validated outcomes measures, including the Knee Injury and Osteoarthritis Outcome Score (KOOS), International Knee Documentation Committee (IKDC) questionnaire, Tegner activity score and Visual Analog Scale (VAS). Both the IKDC and KOOS have demonstrated reliability in patients who undergo ACL reconstruction. Physical examination was also completed, including an assessment of range of motion, Lachman and pivot shift exams, assessment of any residual effusion, and a KT-1000 exam of both the involved and uninvolved knees.

Sixteen patients were identified which met all inclusion and exclusion criteria. 12 patients completed study visits, resulting in 75% follow-up at an average of 2.6 years (range, 1.4-5.1 years) following revision ACL surgery. The summary characteristics of the patients completing the study are provided in Table 1.

TABLE 1
Descriptive Cohort Characteristics

| | |
|---|---|
| n (% follow-up) | 12 (75.0%) |
| Age: mean ± S.D. | 28.1 ± 9.3 years |
| Gender: n (%) male | 6 (50%) |
| BMI: mean ± S.D. | 28.6 ± 6.8 kg/m2 |
| Smoker: n (%) | 2 (17%) |
| Time from index - revision: | 2.9 ± 2.0 years |
| mean ± SD | |
| Initial graft: n (%) autograft | 7 (58%) |
| Revision graft: n (%) | 12 (100%) |
| autograft | |

### Imaging Technique

CT scans were performed using a standard clinical protocol (120-140 keV, auto exposure control, thin slice, bone and soft tissue algorithms) on multidetector CT scanners. Images were reviewed on a clinical Picture Archiving and Communication System (PACS) (Carestream Health, Rochester, N.Y.). CT scans were reviewed in consensus by a fellowship-trained orthopaedic sports surgeon and a fellowship-trained musculoskeletal radiologist. Information was recorded regarding the status of the dowel structure (i.e., intact, fragmented, resorbed, and/or drilled through). The incorporation of the dowels' three-dimensional border into the adjacent native bone was assessed by using an a priori scale (0-24% poor, 25-49% fair, 50-74% good, and 75-100% excellent). incorporation was judged to have occurred when the graft border blended into native bone without intervening non-osseous tissue. Consensus readings were also used to determine the presence or absence of cyst formation or osteolysis.

The dowel size (mm) and dowel relative density in Hounsfield units (HU) within a 50 mm² region of interest was recorded. The Hounsfield unit scale is based upon linear attenuation coefficients and is a scale of relative radiodensity where by definition the radiodensity of distilled water under standard pressure and temperature is zero HU and the radiodensity of air is -1000 HU. Typical HU measurements from the human body are as follows: −120 for fat, 0 to 10 for simple fluid, 40 for muscle, over 150 for light trabecular bone, and 1,000 for cortical bone. When relevant, the purchase of the new interference screws (from the revision surgery) on the dowels by counting screw threads imbedded in the dowel was assessed. Means and standard deviations were calculated to quantify results.

### Surgical Technique

The patient is positioned in the supine position, with use of a lateral post and a bracketed surgical knee holder to aid in hyperflexion of the knee. The contralateral leg may be prepped in the surgical field if graft harvest from the contralateral extremity is planned. Exam under anesthesia followed by standard arthroscopy is performed, with evaluation for any associated injuries. All other injuries are addressed as necessary.

Hardware from the primary surgery is removed unless it is clearly out of the region of planned anatomic location, and graft tissue from the tunnel is debrided. Aggressive debridement of the previous bone tunnels is undertaken, and the tunnels are reamed and dilated with a cannulated system to maintain tunnel axis and compress the recipient cancellous bone. For extremely large tunnels, it may be necessary to use larger cannulated reamers—total joint reamers or femoral INT nail flexible reamers. The tunnel is sized to determine the appropriate size of the allograft bone dowel(s), which are available in a variety of sizes ranging from 10 to 18 mm in diameter and 15-28 mm in length (Cloward dowel, LifeNet Health, Inc., Virginia Beach, Va.). Direct visualization of the tunnel should be performed to ensure that the tunnel is free of unwanted tissue and that the tunnel walls are uncompromised. An appropriately-sized allograft dowel is selected, being the same size as the diameter of the tunnel.

Rehydration of the graft is performed with sterile saline. An appropriately-sized cannulated dilator is placed in the tunnel through an accessory anteromedial portal and a Beath pin is passed through the dilator into the base of the native bone and the dilator is removed leaving the pin centered in the tunnel. The dowel is then pre-drilled along the center of its long axis and placed onto the Beath pin in a cannulated fashion. The dowel is then tamped into place using a cannulated tamp placed over the Beath pin behind the dowel. Due to the press-fit nature of the allograft dowel, new tunnels may then be placed anatomically without regard to previous tunnel position (FIG. 16). The tunnels should, however, attempt to diverge, keeping the entry point of the tunnel as anatomic as possible. Standard graft fixation techniques are used according to surgeon preference.

A standard ACL rehabilitation protocol is utilized postoperatively. Early goals include range of motion, particularly extension and quadriceps rehabilitation. Open chain exercises near full extension are avoided. Patients are allowed to bear weight as tolerated unless a concomitant meniscal repair is performed. Patients are advised that return to unrestricted activity is often delayed by 2-3 months. Running is typically delayed until 3-4 months postoperatively, and return to sports delayed until 6 months postoperatively. Patients are also advised that overall subjective results tend to be inferior to primary ACL reconstruction in order to establish appropriate expectations.

### Results

### Clinical Results

The average KT-1000 of the involved knee was 6.7 mm±2.5 mm and the uninvolved knee was 5.7±2.6 mm, yielding a mean side-to-side difference of 1.0 mm±2.9 mm. 5 patients (42%) had a normal Lachman exam, 6 patients (50%) had a 1+ Lachman and 1 patient (8%) had a 2+ Lachman. 5 patients (42%) had a normal pivot shift examination and 7 patients (58%) had a 1+pivot shift. 10 patients (83%) had no effusion and 2 patients (17%) had a small effusion. On average, the involved knee had 98.4%±2.6% of the range of motion of the uninvolved knee (Table 2).

TABLE 2
Physical Exam

| KT-1000 | Involved Knee: mean ± S.D. | 6.7 mm ± 2.5 mm |
|---|---|---|
| | Uninvolved Knee: mean ± S.D. | 5.7 mm ± 2.6 mm |
| | Side to Side Diff: mean ± S.D. | 1.0 mm |
| Lachman | Normal: # (%) | 5 (42%) |
| | 1+: # (%) | 6 (50%) |
| | 2+: # (%) | 1 (8%) |
| Pivot Shift | Normal: # (%) | 5 (42%) |
| | 1+: # (%) | 7 (58%) |
| | 2+: # (%) | 0 (0%) |
| Knee Effusion | Normal: # (%) | 10 (83%) |
| | 1+: # (%) | 2 (17%) |
| | 2+: # (%) | 0 (0%) |
| Range of Motion | Extension: mean ± S.D. | 0.8 ± 1.9 deg |
| | Flexion: mean ± S.D. | 130.3 ± 5.0 deg |
| | % of Uninvolved Knee: % ± S.D. | 98.4% ± 2.6% |

The average IKDC was 70.2±17.8, the average postoperative Tegner activity score was 4.8±2.8 and the average VAS pain score was 2.8±2.4. The overall average :KOOS was 70.5±22.4. The average KOOS subscale scores were: :KOOS pain: 73.4±22.3, KOOS Symptoms: 67.3±20.0, KOOS ADLs: 82.8±22.9, KOOS Sports 50.8±31,3 and KOOS QOL: 41.2±26.1 (Table 3).

TABLE 3
Patient-Reported Outcome Measures

| | |
|---|---|
| VAS pain | 2.8 ± 2.4 |
| Tegner Activity | 4.8 ± 2.8 |
| IKDC | 70.2 ± 17.8 |
| KOOS Overall | 70.5 ± 22.4 |
| KOOS Pain | 73.4 ± 22.3 |
| KOOS Symptoms | 87.3 ± 20.0 |
| KOOS ADLs | 82.8 ± 22.9 |
| KOOS Sports | 50.8 ± 31.3 |
| KOOS QOL | 41.2 ± 26.1 |

### Imaging Results

Consensus review of all CT scans found that 100% (12/12) dowels had “excellent” (greater than 75%) corporation along their three-dimensional borders with native bone (FIGS. 17 and 18). There were no instances of osteolysis or cyst formation noted. The mean relative density for the 12 dowels was 607.3 HU±105.6 HU.

### Discussion

The present study demonstrates favorable outcomes for single-stage revision ACL reconstruction using an allograft bone dowel for management of an isolated femoral bony deficiency. At an average of 2.6 years' follow-up, patients demonstrated excellent objective outcomes with subjective outcomes similar to those reported for revision ACL surgery. Imaging studies demonstrated excellent incorporation of all bone dowels.

Previous studies have demonstrated subjective outcomes after revision ACL reconstruction to be inferior to primary ACL reconstruction. A recent systematic review of 21 studies including 863 patients who underwent revision ACL reconstruction with a minimum of 2 years' follow-up found a mean IKDC subjective score in 202 patients of 74.8±4.4. (16) The average 1KDC subjective score of our described single-stage technique was 70.2, which is very similar to the findings of the aforementioned systematic review. Data from the MOON cohort revealed similar data, with an average 1KDC of 75.9 for revision ACL reconstruction compared to 83.9 for primary ACL reconstruction. (15) Median KOOS subscale results for revision ACL reconstruction in the MOON cohort were QOL: 62.5, Sports and Recreation: 75, Pain: 83.3. (15) The patients in our study had similar IKDC and KOOS pain scores, but lower KOOS QOL (41.2) and KOOS Sports (50.8). Given the excellent objective examination findings in our cohort, these differences could be related to our smaller study size, as our reported KOOS subscale scores had significant standard deviations. In contrast, our KOOS results were very similar to those reported by Lind, et al: 73±18 for symptoms, 78±17 for pain, 84±16 for activities of daily living, 52±28 for sports, and 48±21 for QOL. (7)

Objective failure of revision ACL reconstruction has been previously defined as a repeat revision procedure, a side-to-side difference of greater than 5 mm measured by a KT-1000 arthrometer or a pivot-shift of grade 2+ or 3+. In the present study, no patients who underwent single-stage revision ACL reconstruction using an allograft bone dowel for an isolated femoral bony defect had an Objective failure. No patients underwent a repeat revision ACL reconstruction. The average side-to-side difference was 1.0 mm (range 0 mm-4.83 mm), demonstrating excellent anteroposterior stability and no objective failure by KT-1000 measurement. 5 patients (42%) had a normal pivot shift examination and 7 patients (58%) had a 1+ pivot shift no patients had a 2+ or 3+ pivot shift Our 0% objective failure rate is better than that reported in a recent systematic review of 863 revision ACL reconstructions of 13.7%±2.7%. A recent study of 827 revision ACL reconstructions in Ontario, Canada found a 4.4% failure rate at an average of 4.8 years postoperatively. (6) While we report a 0% objective failure rate, it is important to note that our data is at an average of 2.6 years' postoperatively.

The described single-stage technique utilizing a dowel for management of bony deficiency, typically for previously malpositioned or widened tunnels, has been previously described. While potentially useful on both the femoral and tibial sides, the present inventors focused the present study on patients with isolated femoral bony defects to create a homogenous study population. There are several advantages to this technique. First, use of an allograft dowel to replenish insufficient bone stock allows uncompromised placement and drilling of tunnels during revision reconstruction. Second, when properly placed, an allograft bone dowel affords sufficient stability to allow use of the surgeons' choice of graft fixation, including interference screws. Alternative single-stage methods for management of bony defects caused by hardware removal, malpositioned tunnels or tunnel enlargement include the use of cylindrical autograft, corticocancellous shims, retention of interference screws or use of a larger interference screws to occupy previous tunnels.

The use of allograft bone is not without associated risks. Although sterilization and sterile handling of allograft have reduced the risk of infection to extremely low rates, viral transmission of hepatitis or human immunodeficiency (HIV) virus remains a concern. Additionally, allograft is known to integrate at a rate slower than autograft and could lead to immunologic responses that interfere with healing. Lastly, the sterilization process may alter the mechanical integrity of the allograft and decrease its structural properties. Despite these risks, the present inventors found excellent incorporation of all grafts on CT imaging and excellent objective stability of the revision ACL grafts.

### Conclusions

A single-stage approach for revision ACL reconstruction utilizing an allograft dowel for isolated femoral bony deficiency yields objective and subjective outcomes comparable to those reported in the literature for two-stage and other single-stage techniques. The allograft bone dowels described in the study demonstrate the ability to integrate with the host bone, adequately fill bony defects from previous tunnels and withstand the stresses required for fixation and stability of ACL grafts.

### ADDITIONAL EXAMPLES

### Example 1

A method for conducting arthroscopic surgery. The method may comprise: inserting a guide wire to a defect site; placing a lumen of a dowel over the guide wire to enable the dowel to travel toward the defect site; placing a guide pin over the guide wire to enable the guide pin to travel along the guide wire to be inserted into the lumen of the dowel; placing the cannula of a tamp over the guide pin to enable the tamp to travel along the guide pin toward the dowel, the tamp having a proximal end and a distal end; and impacting the tamp to apply a force to the proximal end of the tamp wherein the tamp at its distal end impacts the dowel to enable the dowel to advance with a predetermined tit-resistance into the defect site.

### Example 2

The method of example 1, wherein the defect site is a knee.

### Example 3

A tamp device configured for conducting arthroscopic surgery. The tamp device may comprise: a proximal end, distal end, and a cannula longitudinally extending from proximal end to distal end. The tamp is configured wherein its cannula is configured to slide over a guide pin and/or guide wire. The tamp is configured to receive impact force at its proximal end so as transfer the force to a dowel to impact and advance the dowel to a defect site with predetermined fit resistance.

### Example 4

The device of example 3 (as well as subject matter of one or more of any combination of examples 1-2), wherein the defect site is a knee.

### Example 5

The device of example 3 (as well as subject matter of one or more of any combination of examples 1-2 and 4), wherein the distal end of the tamp comprises a flange.

### Example 6

A kit for use in arthroscopic surgery for using a tamp. The kit may comprise: a tamp device comprising a proximal end, distal end, and a cannula longitudinally extending from proximal end to distal end and instructions for use of the tamp. The instructions may include: inserting a guide wire to a defect site, placing a lumen of a dowel over the guide wire to enable the dowel to travel toward the defect site; placing a guide pin over the guide wire to enable the guide pin to travel along the guide wire to be inserted into the lumen of the dowel; placing the cannula of a tamp over the guide pin to enable the tamp to travel along the guide pin toward the dowel; and impacting the tamp to apply a force to the proximal end of the tamp wherein the tamp at its distal end impacts the dowel to enable the dowel to advance with a predetermined fit-resistance into the defect site.

### Example 7

The kit of example 6 (as well as subject matter of one or more of any combination of examples 1-5), wherein the defect site is a knee.

### Example 8

A method for conducting arthroscopic surgery. The method may comprise: placing an implant at a defect site; placing a guide pin to be disposed on the implant; placing the cannula of a tamp over the guide pin to enable the tamp to travel toward the implant, the tamp having a proximal end and a distal end; and impacting the tamp to apply a force to the proximal end of the tamp wherein the tamp at its distal end impacts the implant to enable the implant to advance with a predetermined fit-resistance into the defect site.

### Example 9

The method of example 8 (as well as subject matter of one or more of any combination of examples 1-7), wherein the placing the implant comprises advancing the implant over a guide wire.

### Example 10

The method of example 9 (as well as subject matter of one or more of any combination of examples 1-8), wherein the implant is a dowel.

### Example 11

The method of example 8, wherein the implant is a dowel.

### Example 12

A tamp device configured for conducting arthroscopic surgery. The tamp device may comprise: a proximal end, distal end, and a cannula longitudinally extending from proximal end to distal end. The tamp is configured wherein its cannula is configured to slide over a guide pin and/or guide wire. The tamp is configured to receive impact force at its proximal end so as transfer the force to an implant to impact and advance the impact to a defect site with predetermined fit resistance.

### Example 13

The device of example 12 (as well as subject matter of one or more of any combination of examples 1-11), wherein the implant is a dowel or surgical hardware or material.

### Example 14

A kit for use in arthroscopic surgery for using a tamp. The kit may comprise: a tamp device comprising a proximal end, distal end, and a cannula longitudinally extending from proximal end to distal end, and instructions for use of the tamp. The instructions may include: placing an implant at a defect site; placing a guide pin to be disposed on the implant; placing the cannula of a tamp over the guide pin to enable the tamp to travel toward the implant; and impacting the tamp to apply a force to the proximal end of the tamp wherein the tamp at its distal end impacts the implant to enable the implant to advance with a predetermined fit-resistance into the defect site.

### Example 15

The kit of example 14 (as well as subject matter of one or more of any combination of examples 1-13), wherein the placing the implant comprises advancing the implant over a guide wire.

### Example 16

The kit of example 15 (as well as subject matter of one or more of any combination of examples 1-14), wherein the implant is a dowel.

### Example 17

The kit of example 14 (as well as subject matter of one or more of any combination of examples 1-13 and 15-16), wherein the implant is a dowel.

### Example 18

The method of using or selling any of the devices (structures or related systems and devices) or their components (in whole or in part) provided in any one or more of examples 1-17.

### Example 19

The method of manufacturing any of the devices (structures or related systems and devices) or their components (in whole or in part) provided in any one or more of examples 1-17.

### REFERENCES

The devices, systems, apparatuses, structures, compositions, materials, kits, and methods of various embodiments of the invention disclosed herein may utilize aspects disclosed in the following references, applications, publications and patents and which are hereby incorporated by reference herein in their entirety (and which are not admitted to be prior art with respect to the present invention by inclusion in this section):

1. Mayr R, Rosenberger R, Agraharam D, Smekal V, El Attal R, Revision anterior cruciate ligament reconstruction: an update. Arch Orthop Trauma Surg 2012 September; 132(9):1299-313.

2. Mall N A, Chalmers P N, Moric M, Tanaka M J, Cole B J, Bach B R, Jr., Paletta G A, Jr. Incidence and Trends of Anterior Cruciate Ligament Reconstruction in the United States, AM J Sports Med 2014 Aug. 1.

3. Wasserstein D, Khoshbin A, Dwyer T, Chahal J, Gandhi R, Mahomed N, Ogilvie-Harris D. Risk factors for recurrent anterior cruciate ligament reconstruction: a population study in Ontario, Canada, with 5-year follow-up. Am J Sports Med 2013 September; 41(9):2099-107.

4. Lyman S, Koulouvaris P, Sherman S, Do H, Mandl L A, Marx R G. Epidemiology of anterior cruciate ligament reconstruction: trends, readmissions, and subsequent knee surgery. J Bone Joint Surg Am 2009 October; 91(10):2321-8.

5. Barber F A, Cowden C H, 3rd, Sanders E J. Revision rates after anterior cruciate ligament reconstruction using bone-patellar tendon-bone allograft or autograft in a population 25 years old and younger. Arthroscopy 2014 April; 30(4):483-91.

6. Leroux T, Wasserstein D, Dwyer T, Ogilvie-Harris D J, Marks P H, Bach B R, Jr., Townley J B, Mohamed N, Chahal J. The Epidemiology of Revision Anterior Cruciate Ligament Reconstruction in Ontario, Canada. Am J Sports Med 2014 Sep. 11.

7. Lind M, Menhert F, Pedersen A B. Incidence and outcome after revision anterior cruciate ligament reconstruction: results from the Danish registry for knee ligament reconstructions, Am J Sports Med 2012 July; 40(7):1551-7.

8. Kamien P M, Hydrick J M, Replogle W H, Go L T, Barrett G R. Age, graft size, and Tegner activity level as predictors of failure in anterior cruciate ligament reconstruction with hamstring autograft. Am J Sports Med 2013 August; 41(8):1808-12.

9. Magnussen R A, Lawrence J T, West R L, Toth A P, Taylor D C, Garrett W E. Graft size and patient age are predictors of early revision after anterior cruciate ligament reconstruction with hamstring autograft. Arthroscopy 2012 April; 28(4):526-31.

10, Mariscalco M W, Flanigan D C, Mitchell J, Pedroza A D, Jones M H, Andrish J T, Parker R D, Kaeding C C, Magnussen R A. The influence of hamstring autograft size on patient-reported outcomes and risk of revision after anterior cruciate ligament reconstruction: a Multicenter Orthopaedic Outcomes Network (MOON) Cohort Study. Arthroscopy 2013 December; 29(12): 1948-53.

11. Park S Y, Oh H, Park S, Lee J H, Lee S H, Yoon K H. Factors predicting hamstring tendon autograft diameters and resulting failure rates after anterior cruciate ligament reconstruction. Knee Surg Sports Traumatol Arthrosc 2013 May; 21(5).1111-8.

12. Ahlden M. Samuelsson K, Semert N, Forssblad M, Karlsson J, Kartus J. The Swedish National Anterior Cruciate Ligament Register: a report on baseline variables and outcomes of surgery for almost 18,000 patients. Am J Sports Med 2012 October; 40(10):2230-5.

13. Hettrich C M, Dunn W R, Reinke E K, MOON Group, Spindler K P. The rate of subsequent surgery and predictors after anterior cruciate ligament reconstruction: two- and 6-year follow-up results from a multicenter cohort. Am J Sports Med 2013 July; 41(7):1534-40.

14. Maletis G B, Inacio M C, Funahashi T T. Analysis of 16,192 anterior cruciate ligament reconstructions from a community-based registry. Am J Sports Med 2013 September; 41(9):2090-8.

15. Wright R, Spindler K, Huston L, Amendola A, Andrish J, Brophy R, Carey J, Cox C, Flanigan D, Jones M, Kaeding C, Marx R, Matava M, McCarty E, Parker R, Vidal A, Wolcott M, Wolf B. Dunn W. Revision ACL reconstruction outcomes: MOON 271 cohort. J Knee Surg 2011 December; 24(4):289-94.

16. Wright R W, Gill C S, Chen L, Brophy R H, Matava M J, Smith M V, Mall N A. Outcome of revision anterior cruciate ligament reconstruction: a systematic review. J Bone Joint Surg Am 2012 Mar. 21; 94(6):531-6.

17, MARS Group, Wright R W, Huston L J, Spindler KP, Dunn W R, Haas A K, Allen C R, Cooper D E, DeBerardino T M, Lantz B B, Mann B J, Stuart M J. Descriptive epidemiology of the Multicenter ACL Revision Study (MARS) cohort. Am J Sports Med 2010 October; 38(10):1979-86.

18. Marchant B G, Noyes F R, Barber-Westin S D, Fleckenstein C. Prevalence of nonanatomical graft placement in a series of failed anterior cruciate ligament reconstructions. Am J Sports Med 2010 October; 38(10):1987-96.

19. Conte E J, Hyatt A E, Gatt C J Jr., Dhawan A. Hamstring autograft size can be predicted and is a potential risk factor for anterior cruciate ligament reconstruction failure. Arthroscopy 2014 July; 30(7).882-90.

20. Denti M, Lo Vetere D, Bait C, Schonhuber H, Melegati G, Volpi P. Revision anterior cruciate ligament reconstruction: causes of failure, surgical technique, and clinical results. Am J Sports Med 2008 October; 36(10).1896-902.

21. George M S, Dunn W R, Spindler K P. Current concepts review: revision anterior cruciate ligament reconstruction. Am J Sports Med 2006 December; 34(12).2026-37.

22. Gifstad T, Foss O A, Engebretsen L, Lind M, Forssblad M, Albrektsen G, Drogset J O. Lower Risk of Revision With Patellar Tendon Autografts Compared With Hamstring Autografts: A Registry Study Based on 45,998 Primary ACL Reconstructions in Scandinavia. Am J Sports Med 2014 Sep. 8.

23. Kamath G V, Redfern J C, Greis P E, Burks R T. Revision anterior cruciate ligament reconstruction. Am J Sports Med 2011 January; 39(1).199-217.

24. Webster K E, Feller J A, Leigh W B, Richmond A K. Younger patients are at increased risk for graft rupture and contralateral injury after anterior cruciate ligament reconstruction. Am J Sports Med 2014 March; 42(3):641-7.

25. Noyes F R, Barber-Westin S D. Revision anterior cruciate surgery with use of bone-patellar tendon-bone autogenous grafts. J Bone Joint Surg Am 2001 August; 83-A(8):1131-43.

26. Coats A C, Johnson D L. Two-stage revision anterior cruciate ligament reconstruction: indications, review, and technique demonstration. Orthopedics 2012 November; 35(11).958-60.

27. Barrett G R, Brown T D. Femoral tunnel defect filled with a synthetic dowel graft for a single staged revision anterior cruciate ligament reconstruction. Arthroscopy 2007 July; 23(7):796.e1,796,e4.

28. Franceschi F, Papalia R, Di Martino A, Rizzello G, Ailaire R, Denaro V. A new harvest site for bone graft in anterior cruciate ligament revision surgery. Arthroscopy 2007 May; 23(5):558.e1,558.e4.

29. Niki Y. Matsumoto H, Enomoto H, Toyama Y, Suda Y. Single-stage anterior Cruciate ligament revision with bone-patellar tendon-bone: a case-control series of revision of failed synthetic anterior cruciate ligament reconstructions. Arthroscopy 2010 August; 26(8):1058-65.

30. Azar F M. Revision anterior cruciate ligament reconstruction. Instr Course Lect 2002; 51:335-42.

31. Allen C R, Giffin J R, Harner C D. Revision anterior cruciate ligament reconstruction. Orthop Clin North Am 2003 January; 34(1):79-98.

32. Getelman M H, Friedman M J. Revision anterior cruciate ligament reconstruction surgery. J Am Acad Orthop Surg 1999 May-June; 7(3):189-98.

33. Noyes F R, Barber-Westin S D. Revision anterior cruciate ligament reconstruction: report of 11-year experience and results in 114 consecutive patients. Instr Course Lect 2001; 50:451-61.

34. Said H G, Baloch K, Green M. A new technique for femoral and tibial tunnel bone grafting using the OATS harvesters in revision anterior cruciate ligament reconstruction. Arthroscopy 2006 July; 22(7):796.e1,796.e3.

35. Safran M R, Harner C D. Technical considerations of revision anterior cruciate ligament surgery. Clin Orthop Relat Res 1996 April; (325)(325):50-64.

36. Ra H J, Ha J K, Kim J G. One-stage revision anterior cruciate ligament reconstruction with impacted bone graft after failed primary reconstruction. Orthopedics 2013 November; 36(11):860-3.

37. Hambly K, Griva K. IKDC or KOOS: which one captures symptoms and disabilities most important to patients who have undergone initial anterior cruciate ligament reconstruction? Am J Sports Med 2010 July; 38(7):1395-404.

38. Sgaglione N A, Miniaci A. Gillogly S D, Carter T R. Update on advanced surgical techniques in the treatment of traumatic focal articular cartilage lesions in the knee. Arthroscopy 2002 February; 18(2 Suppl 1):9-32.

39. Miller M D. Revision cruciate ligament surgery with retention of femoral interference screws. Arthroscopy 1998 January-February; 14(1):111-4.

40. Battaglia, Todd C., et al., “Management of Bony Deficiency in Revision Anterior Cruciate Ligament Reconstruction Using Allograft Bone Dowels: Surgical Technique”, Arthroscopy: The Journal of Arthroscopic and Related Surgery, Vol. 21, No. 6 (June), 2005: pp. 767.e1-767.e5.

41. Gilmore, Carl J., et al., “ACL Revision Reconstruction: Results of a Novel Single Stage Approach Using Allograft Dowel Bone Grafting”, The Orthopaedic Journal of Sports Medicine, 1 (4) (Suppl. 1), DOI: 10.1177/2325967113S00072, 2013.

42. Hamann, Josh, and Miller, Mark D., “Single Stage ACL Revision Reconstruction: Indications and Technique”, Chapter 12, pp. 119-126, R. G. Marx (fed.), Revision ACL Reconstruction: Indications and Technique, DOI 10.1007/978-1-4614-0766-9_12, Springer Science Business Media New York 2014.

43. Miller, Mark D., et al., “Procedure 2-Revision ACL Reconstruction”, Complex (Double-bundle and Revision) Anterior Cruciate Ligament Reconstruction, Sports Knee Surgery, 2008, pp 299-313.

44, U.S. Pat. No. 8,535,377 B2, Myers, et “Double Bundle CL Repair System”, Sep. 17, 2013.

45. International Patent Application No. WO 98/22047, Feeney, M., et al., “Bone Implant”, May 28, 1998.

46. U.S. Pat. No. 5,601,562, Wolf, E., et al., “Forked Insertion Tool and Method of Arthroscopic Surgery Using the Same”, Feb. 11, 1997.

47. U.S. Pat. No. 5,320,115, Kenna, R., “Method and Apparatus for Arthroscopic Knee Surgery”, Jun. 14, 1994.

48. U.S. Pat. No. 6,001,100, Sherman, M., et al., “Bone Block Fixation Implant”, Dec. 14, 1999.

49. U.S. Patent Application Publication No. US 2009/0018654 A1, Schmieding, R., et al., “Double Socket ACS Reconstruction”, Jan. 15, 2009.

50. U.S. Pat. No. 7,686,838 B2, Wolf, A., et al., “External Bullet Anchor Apparatus and Method for Use in Surgical Repair of Ligament or Tendon”, Mar. 30, 2010.

51, U.S. Pat. No. 5,919,196, Bobic, v., ET AL., “Method and Apparatus for Osteochondral Autograft Transplantation”, Jul. 6, 1999.

52. U.S. Pat. No. 8,663,324 B2, Schmieding, et al., “Double Socket ACL Reconstruction”, Mar. 4, 2014.

53. U.S. Pat. No. 6,699,252 B2, Farr, II, J., et al., “Methods and Instruments for Improved Meniscus Transplantation”, Mar. 2, 2004.

54. U.S. Patent Application Publication No. US 2012/0059469, A1, Myers, T., et al., “Double Bundle ACS Repair System”, Mar. 8, 2012.

55. U.S. Pat. No. 5,785,714, Morgan, C., et al., “Method of ACL Reconstruction Using Double Socket Graft Placement and Fixation”, Jul. 28, 1988.

56. U.S. Pat. No. 8,048,079 B2, Iannarone, R, “Retrograde Cutting Instrument”, Nov. 1, 2011.

57. U.S. Pat. No. 5,603,716, Morgan, C., et al., “Method of Ligamen econstruction Using Double Socket Graft Placement and Fixation”, Feb. 18, 1997.

58. U.S. Pat. No. 5,152,790, Rosenberg, T., et al, “Ligament Reconstruction Graft Anchor Apparatus”, Oct. 6, 1992.

59, European Patent Application Publication No. EP 0824893 A2, Bobic, V., et al, “Apparatus for Osteochondral Autograft Transplantation”, Feb. 25, 1998.

Unless clearly specified to the contrary, there is no requirement for any particular described or illustrated activity or element, any particular sequence or such activities, any particular size, speed, material, duration, contour, dimension or frequency, or any particularly interrelationship of such elements, Moreover, any activity can be repeated, any activity can be performed by multiple entities, and/or any element can be duplicated. Further, any activity or element can be excluded, the sequence of activities can vary, and/or the interrelationship of elements can vary. It should be appreciated that aspects of the present invention may have a variety of sizes, contours, shapes, compositions and materials as desired or required.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict: that would render invalid any claim herein or seeking priority hereto, then any such conflicting information in such incorporated by reference material is specifically not incorporated by reference herein.

We claim:

1. A method for conducting arthroscopic surgery, said method comprising:
inserting a guide wire to a defect site;
placing a lumen of a dowel over the guide wire to enable said dowel to travel toward the defect site;
placing a guide pin over said guide wire to enable said guide pin to travel along said guide wire to be inserted into the lumen of said dowel;
placing the cannula of a tamp over the guide pin to enable said tamp to travel along said guide pin toward the dowel, said tamp having a proximal end and a distal end; and
impacting said tamp to apply a force to said proximal end of said tamp wherein said tamp at its distal end impacts the dowel to enable the dowel to advance with a predetermined fit-resistance into the defect site.

2. The method of claim 1, wherein the defect site is a knee.

3. A tamp device configured for conducting arthroscopic surgery, said tamp device comprising:
a proximal end, distal end, and a cannula longitudinally extending from proximal end to distal end, wherein:
said tamp is configured wherein it's cannula is configured to slide over a guide pin and/or guide wire; and
said tamp is configured to receive impact force at its proximal end so as transfer the force to a dowel to impact and advance the dowel to a defect site with predetermined fit resistance.

4. The device of claim 3, wherein the defect site is a knee.

5. The device of claim 3, wherein said distal end of said tamp comprises a flange.

6. A kit for use in arthroscopic surgery for using a tamp, said kit comprising:
a tamp device comprising a proximal end, distal end, and a cannula longitudinally extending from proximal end to distal end, and
instructions for use of said tamp including:
inserting a guide wire to a defect site;
placing a lumen of a dowel over the guide wire to enable said dowel to travel toward the defect site;
placing a guide pin over said guide wire to enable said guide pin to travel along said guide wire to be inserted into the lumen of said dowel;
placing the cannula of a tamp over the guide pin to enable said tamp to travel along said guide pin toward the dowel; and
impacting said tamp to apply a force to said proximal end of said tamp wherein said tamp at its distal end impacts the dowel to enable the dowel to advance with a predetermined fit-resistance into the defect site.

7. The kit of claim 6, wherein the defect site is a knee.

8. A method for conducting arthroscopic surgery, said method comprising:
placing an implant at a defect site;
placing a guide pin to be disposed on said implant;
placing the cannula of a tamp over the guide pin to enable said tamp to travel toward the implant, said tamp having a proximal end and a distal end; and
impacting said tamp to apply a force to said proximal end of said tamp wherein said tamp at its distal end impacts the implant to enable the implant to advance with a predetermined fit-resistance into the defect site.

9. The method of claim 8, wherein said placing said implant comprises advancing said implant over a guide wire.

10. The method of claim 9, wherein said implant is a dowel.

11. The method of claim 8, wherein said implant is a dowel.

12. A tamp device configured for conducting arthroscopic surgery, said tamp device comprising:
a proximal end, distal end, and a cannula longitudinally extending from proximal end to distal end, wherein:
said tamp is configured wherein its cannula is configured to slide over a guide pin and/or guide wire; and
said tamp is configured to receive impact force at its proximal end so as transfer the force to an implant to impact and advance the impact to a defect site with predetermined fit resistance.

13. The device of claim 12, wherein said implant is a dowel or surgical hardware or material.

14. A kit for use in arthroscopic surgery for using a tamp, said kit comprising:
a tamp device comprising a proximal end, distal end, and a cannula longitudinally extending from proximal end to distal end, and
instructions for use of said tamp including:
placing an implant at a defect site;
placing a guide pin to be disposed on said implant;
placing the cannula of a tamp over the guide pin to enable said tamp to travel toward the implant; and
impacting said tamp to apply a force to said proximal end of said tamp wherein said tamp at its distal end impacts the implant to enable the implant to advance with a predetermined fit-resistance into the defect site.

15. The kit of claim 14, wherein said placing said implant comprises advancing said implant over a guide wire.

16. The kit of claim 15, wherein said implant is a dowel.

17. The kit of claim 14, wherein said implant is a dowel.
